(19) United States
(12) Patent Application Publication
(10) Pub. No.: US 2020/0390951 A1
(43) Pub. Date: Dec. 17, 2020

(54) Intravenous System Including Pump, Vascular Access Device And Securement Device And Methods Thereof

(71) Applicant: Bard Access Systems, Inc., Salt Lake City, UT (US)

(72) Inventors: Daniel B. Blanchard, Bountiful, UT (US); Todd P. McFarland, Larue, TX (US); Glade H. Howell, Draper, UT (US)

(21) Appl. No.: 16/888,277

(22) Filed: May 29, 2020

Related U.S. Application Data
(60) Provisional application No. 62/860,100, filed on Jun. 11, 2019.

(51) Int. Cl. A61M 1/10; A61M 1/30; A61M 1/36

(52) U.S. Cl. CPC A61M 1/1086; A61M 1/301; A61M 1/3661; A61M 1/1039; A61M 2205/3334; A61M 2230/06; A61M 2205/3553; A61M 2230/201; A61M 2230/50; A61M 2205/50; A61M 2205/3561; A61M 2205/3569; A61M 2230/208

(57) ABSTRACT

Embodiments disclosed herein are directed to a system including an intravenous (IV) pump, a vascular access device (VAD), securement devices, and methods thereof. The intravenous (IV) pump, vascular access device (VAD), and securement device can include a sensor, printed circuit board (PCB), communication module, and the like to determine the characteristics of the VAD device in use, the fluid being administered, and physiological characteristics of the patient. The pump unit can utilize this information to modify the flow characteristics of the fluid being administered. The VAD characteristics can be predetermined and stored on the VAD, derived from sensors, or derived from remote databases using a unique identifier. Fluid and physiological characteristics can be determined from sensors located on the pump, VAD, securement device or combinations thereof.

### PRIORITY

This application claims the benefit of priority to U.S. Patent Application No. 62/860,100, filed Jun. 11, 2019, which is incorporated by reference in its entirety into this application.

### BACKGROUND

Most intravenous (IV) delivery is by way of an active pump or relies on passive gravity flow. The Vascular Access Device (VAD) connected to these pumps or gravity flow systems can include peripheral IV catheter, midline catheter, Peripherally Inserted Central Catheter (PICC), acute or chronic Central Venous Catheter (CVC), and the like. These different Vascular Access Devices display different fluid-mechanical characteristics both between and within categories. Differences in device size, length, lumen configuration, and the like can all have an effect on the fluid mechanics, or flow characteristics, of the system as a whole. Currently, IV pumps do not adequately account for these differences in fluid mechanics, nor do the pumps promote proper maintenance of a VAD.

What is needed, therefore is a system and a method that includes a pump, a VAD, and a securement device. Characteristics of the system are provided to the pump unit so that the pump can modify output to accommodate different flow characteristics. The different system characteristics can include the particular type of VAD in use, the fluid being administered, patient physiology, and the like. The system characteristics can be detected and provided to the pump unit by sensors in the securement device, the VAD, the pump itself, or combinations thereof.

### SUMMARY

Briefly summarized, embodiments disclosed herein are directed to a system including an intravenous (IV) pump, vascular access device (VAD), and securement device and methods thereof. The intravenous (IV) pump, vascular access device (VAD) and securement device can include a sensor, printed circuit board (PCB), communication module, and the like to determine the characteristics of the VAD device in use, the fluid being administered, and physiological characteristics of the patient. The pump unit then uses this information to modify the flow characteristics of the fluid being administered.

The VAD characteristics can be predetermined and stored on the VAD, derived from sensors, or derived from remote databases using a unique identifier. The VAD characteristics can include dimensions such as length, size, gauge, number of lumens, lumen configuration, cross-sectional area, cross-sectional shape of the VAD, or components thereof, and details of which can be stored on the VAD itself. The VAD characteristics can also include data derived from sensors located on the VAD that can be used to determine one or more of the above aspects of the VAD. The VAD characteristics can also include make, model, batch number, serial number, or unique identifier of the specific type of VAD, or components thereof. These identifying details can be stored on the VAD itself and queried against a database to retrieve one or more of the characteristics of the VAD.

The sensors and PCBs can be located on any component of the VAD. For example, a sensor and PCB could be located in a needle-less injection cap. In one embodiment, a sensor is positioned in extension tubing that connects the pump to the VAD.

The characteristics of the fluid being administered (“fluid characteristics”) can include fluid type (e.g. drug type, Ringers solution, saline, etc.), volume, concentration of particular ingredients (active ingredients, non-active ingredients, etc.), pH, viscosity, density, and the like.

Physiological characteristics of the patient can include heart rate, ECG, oxygen saturation, blood pressure, core body temperature, blood glucose level, lactate level, and the like.

The flow characteristics, or fluid mechanics, of the fluid being administered include properties relating of the movement of the fluid being administered and can include flow rate, change in flow rate, pressure, change in pressure, back pressure, and the like.

In an aspect of the invention, the pump unit can modify the fluid mechanics based on predefined settings, or “modes,” in response to predefined characteristics. For example, the pump can modify performance based on the specific characteristics of the VAD attached thereto.

In an aspect of the invention, the pump unit can adapt the fluid mechanics in response to changes using a continuous feedback loop.

Disclosed herein is an infusion system, comprising a vascular access device including one of a first sensor and a first printed circuit board (PCB), a securement device including one of a second sensor and a second PCB, the securement device configured for securing the vascular access device to a skin surface of a patient, and a pump unit in fluid communication with the vascular access device. The pump unit communicatively coupled with one of the vascular access device and the securement device and receiving information therefrom to modify a flow characteristic of a fluid disposed therein.

In some embodiments, the information received from one of the vascular access device and the securement device includes one of a vascular access device characteristic, fluid characteristics, and physiological characteristic. The vascular access device characteristic includes one of sensor data and a unique identifier. The pump unit queries the unique identifier against a database to retrieve additional vascular access device characteristics. The flow characteristic of the fluid includes one of flow rate, change in flow rate, and pressure. The first and second sensor detects one of body temperature, heart rate, fluid pressure, pH, glucose, and lactate. One of the first and second sensor includes an array of two or more sensors. The pump unit includes a power source operably connected to one of the first sensor, second sensor, first PCB, and second PCB. The pump unit is communicatively coupled with one of a remote device and network for sending and receiving information about one of the VAD characteristics, fluid characteristics, physiological characteristics, and flow characteristics.

Also disclosed is a pump unit for providing a fluid to a vasculature of a patient, the pump unit comprising a pump, a sensor for detecting a fluid characteristic of the fluid, a printed circuit board (PCB) communicatively coupled with a vascular access device. The pump unit designed to retrieve information from the vascular access device and the sensor to determine a flow characteristic of the fluid.

In some embodiments, the flow characteristic includes one of flow rate, change in flow rate, and pressure. The fluid characteristic includes one of a fluid type, volume, concentration, pH, and viscosity. The pump unit further includes a securement device for securing an external portion of the vascular access device to a skin surface of the patient, the securement device communicatively coupled with the vascular access device and including a sensor for detecting a physiological characteristic of the patient. The pump unit provides flow characteristic information to a remote location, the remote location including one of a handheld device, smartphone, laptop computer, server, storage device, patient electronic medical records system, and nurse station. The pump unit includes a continuous feedback loop to modify the flow characteristic of the fluid in response to a change in information from one of the vascular access device and the sensor.

Also disclosed is a method of providing dialysis to a patient, the method comprising, providing a pump unit, a vascular access device, and a securement device, one of the vascular access device and the securement device including a sensor, accessing a vasculature of a patient using the vascular access device, securing an external portion of the vascular access device to a skin surface of the patient using the securement device, providing power to the vascular access device from a power source located on the pump unit, deriving a vascular access device characteristic from the vascular access device, deriving a fluid characteristic of the fluid from a sensor included in the pump unit, determining a flow characteristic of the fluid, and modifying a pump output according to the flow characteristic of the fluid.

In some embodiments, the method further includes a continuous feedback loop wherein in response to a change in one of the vascular access device characteristic and the fluid characteristic, the pump unit modifies the flow characteristic of the fluid. The securement device includes a sensor for detecting a physiological characteristic of the patient, the physiological characteristic including one of heart rate, ECG, oxygen saturation, blood pressure, core body temperature, blood glucose level, and lactate level. Wherein in response to a change in one of the vascular access device characteristic, the fluid characteristic, and the physiological characteristic of the patient the pump unit modifies the flow characteristic of the fluid.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A-1B shows an intravenous pump system including a VAD, securement device, and associated remote device in accordance with an embodiment of the disclosure;

FIG. 2 shows a schematic view of a pump in accordance with an embodiment of the disclosure;

FIGS. 3A-3B shows embodiments of vascular access devices in accordance with the disclosure;

FIGS. 4A-4B show plan views of an embodiment of a securement device in accordance with the disclosure;

FIG. 4C shows a side view of an embodiment of a securement device in accordance with the disclosure;

FIGS. 5A-5B show plan views of an embodiment of a securement device in accordance with the disclosure;

FIG. 5C shows a side view of an embodiment of a securement device in accordance with the disclosure;

FIG. 6 shows a schematic view of an intravenous pump system including a VAD and securement device in accordance with an embodiment of the disclosure; and

FIG. 7 shows a flow diagram of information flow within an intravenous pump system in accordance with an embodiment of the disclosure.

### DETAILED DESCRIPTION

Reference will now be made to figures wherein like structures will be provided with like reference designations. It is understood that the drawings are diagrammatic and schematic representations of exemplary embodiments of the present invention, and are neither limiting nor necessarily drawn to scale.

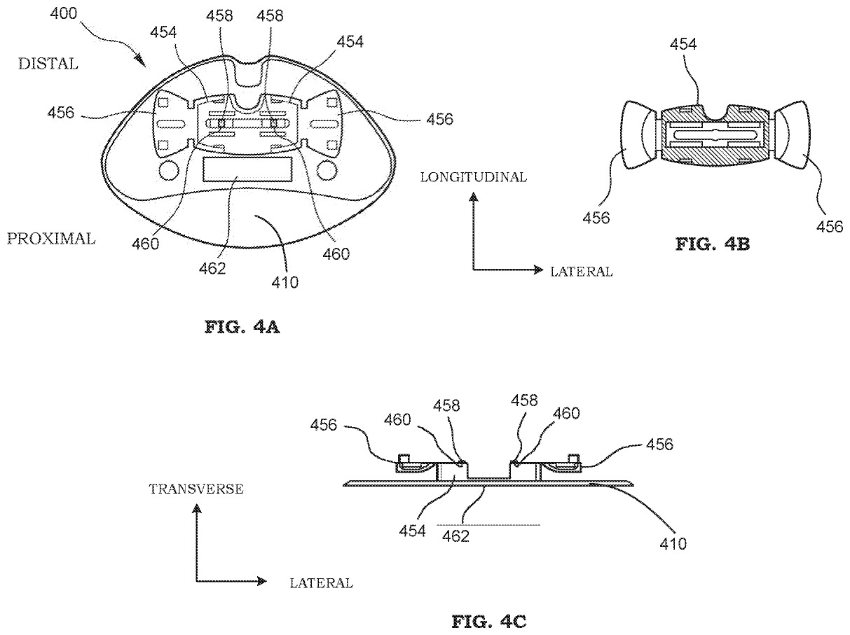

To assist in the description of the securement system, the following coordinate terms are used (see FIGS. 4A-C). A “longitudinal axis” is generally parallel to the axis of a catheter of the device. A “lateral axis” is normal to the longitudinal axis. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. The term “axial” as used herein refers to the axis of the catheter, and therefore is substantially synonymous with the term “longitudinal” as used herein.

For clarity it is to be understood that the word “proximal” refers to a direction relatively closer to a clinician using the device to be described herein, while the word “distal” refers to a direction relatively further from the clinician. For example, the tip of the catheter placed within the body of a patient is considered a distal end of the device, while the catheter hub remaining outside the body is towards a proximal end of the device. Also, the words “including,” “has,” and “having,” as used herein, including the claims, shall have the same meaning as the word “compri sing.”

The terms “upper,” “lower,” “top,” “bottom,” “underside,” “upperside” and the like, which also are used to describe the present securement system, are used in reference to the illustrated orientation of the embodiment. For example, the term “upperside” is used to describe the side of the device that is located above a lateral axis that passes through the axis of the catheter. The term “underside” is used to describe the portion of the device that is located below a lateral axis that passes through the axis of the catheter. The terms “left” and “right” are used consistently throughout the disclosure and are used to describe structures from the perspective of the clinician using the device.

Briefly summarized, embodiments herein are generally directed to a system including an intravenous (IV) pump, vascular access device (VAD), and securement device and methods thereof. Embodiments include a pump unit communicatively couple with a VAD and optionally a securement device to retrieve information from the sensor and printed circuit board (PCB) located thereon. The pump unit can then use this information to modify the flow characteristics of a fluid passing therethrough. Embodiments herein further describe additional aspects of the system and methods of use thereof.

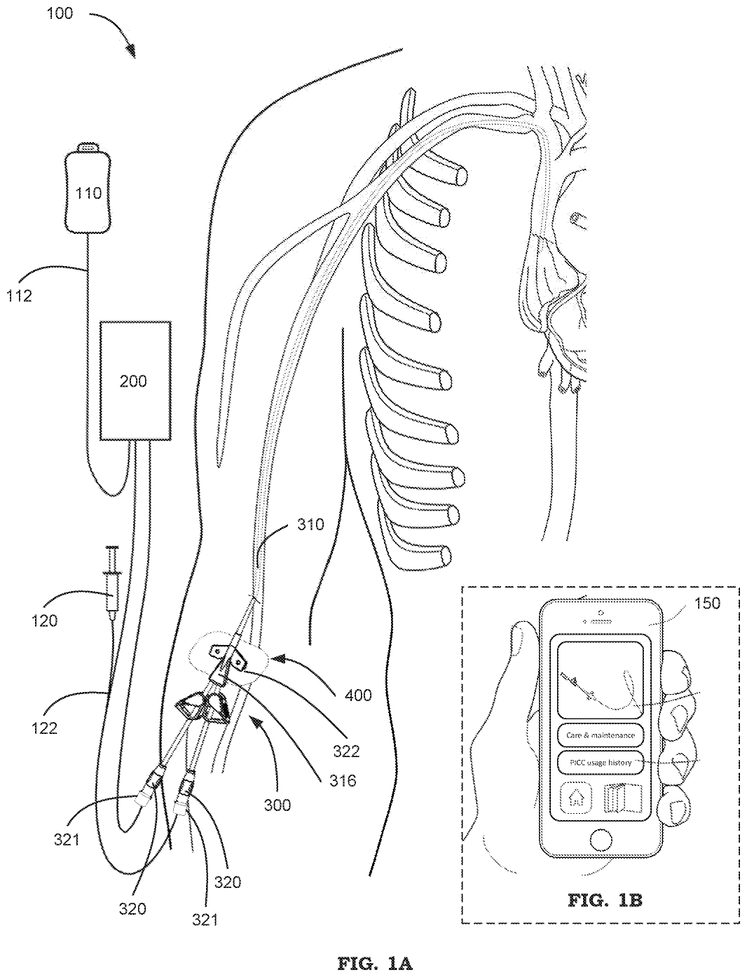

FIG. 1 depicts an exemplary embodiment of an intravenous pump system (“system”) 100. The system 100 includes an IV pump unit 200, a Vascular Access Device (VAD) 300, and a securement device 400. Although a dual lumen PICC VAD is shown in FIG. 1, it will be appreciated that other types of VAD can be used with the system 100 as discussed herein, including, for example, including catheters, needles, port catheters, and the like. The pump unit 200 enables fluid movement through the system 100. A fluid drip assembly 110 is also included to provide fluid to the system 100, by way of the pump unit 200 and supply lines 112. Optionally, a syringe 120 is included to provide an additional fluid inlet into a corresponding supply line 122. This allows a clinician to administer additional fluids, for example medications. The VAD 300 includes a catheter 310, a distal portion thereof being disposed within a vasculature of a patient. A proximal end of the VAD 300 includes a connector 320 for fluidly connecting the VAD 300 with system 100 by way of supply lines 112, 122. The connector 320 can include a needle-less injection cap 321, such as BD MAXPLUS™, BD MAXZERO™, and NEUTRACLEAR™. The VAD 300 can further include securement features 322 that co-operate with the securement device 400 for securing an external portion of the VAD 300 to a skin surface of the patient. The VAD 300 can also include a hub 316. In an embodiment, the system 100 can be communicatively coupled with a remote device 150. As shown in FIG. 1B, the remote device 150 can include a handheld device, although other remote devices such as smartphones, laptop computers, servers, storage devices, patient electronic medical records systems, and nurse stations are contemplated, as will be discussed herein.

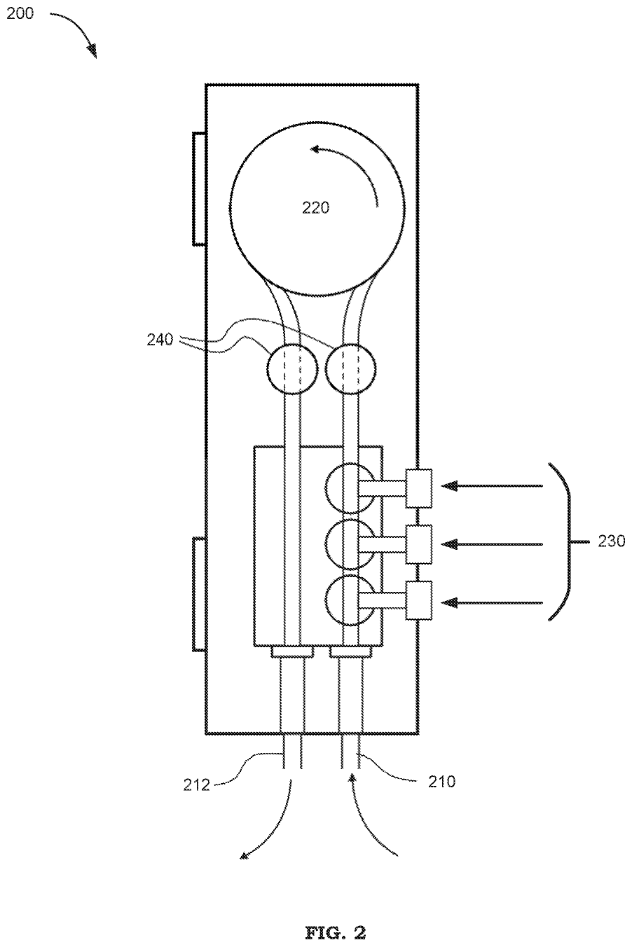

FIG. 2 depicts further details of the pump unit 200 of FIG. 1, including a fluid inlet 210 and a fluid outlet 212 that are configured to fluidly communicate with corresponding supply lines 122 (FIG. 1). In an embodiment, the system 100 can be used as part of a dialysis system to circulate blood through a dialysis machine. As such the inlet 210 can receive a blood supply from the patient and the outlet 212 can provide a blood return. In an embodiment, the system 100 can be used as part of a pump driven intravenous infusion system which provides a fluid, saline solution, blood, medications, or the like, to the vasculature of the patient. As such the inlet 210 can receive an infusion fluid from a supply, such as drip assembly 110 and the outlet 212 can provide the infusion fluid to the VAD 300.

A pump 220 is included in the pump unit 200 to cause the movement of the fluid. Additionally, various input ports 230 are included on the pump unit 250 in fluid communication with the fluid inlet 210 to enable additional fluids to be provided, including heparin, saline, arterial input, or the like. One or more sensors 240 are also included in the pump unit 200 and arranged so as to measure one or more characteristics of the fluid. Examples of such sensors include a glucose meter, oxygen sensor, lactic acid sensor, cardiac output sensor, hematocrit sensor, electrolyte sensor, or the like. The location of the sensors 240 can vary from what is shown. Advantageously, disposal of the sensors 240 in the pump unit 200, as opposed to the on the VAD 300 itself, enables sensors of relatively greater size to be employed without unduly increasing the size of the VAD 300 or securement device 400.

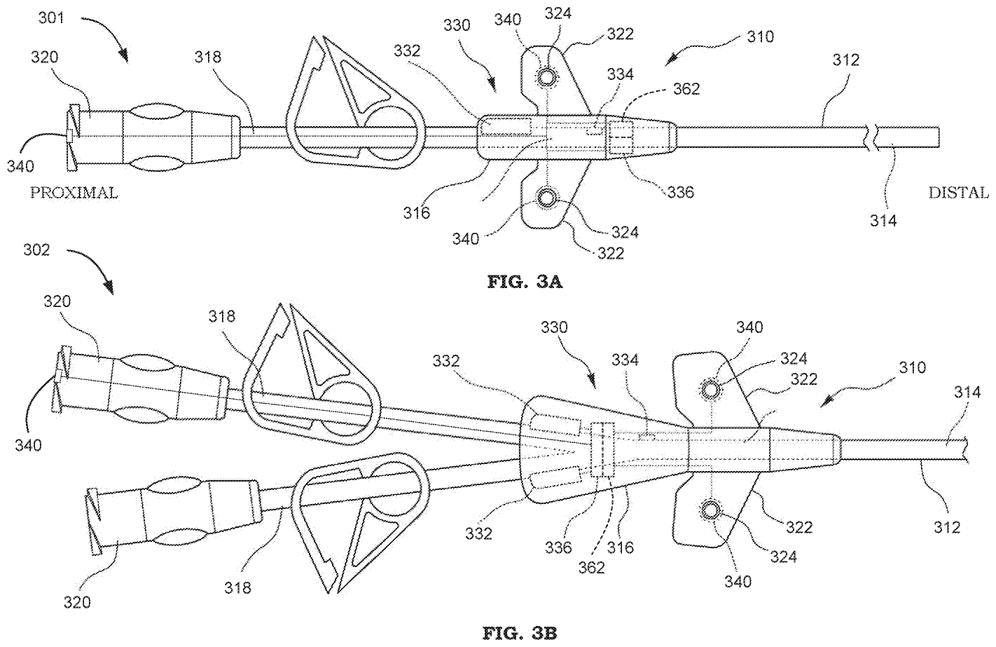

FIGS. 3A-3B show exemplary embodiments of a VAD 300. FIG. 3A shows an embodiment of a single lumen VAD 301 and FIG. 3B shows an exemplary embodiments of dual-lumen VAD 302. The VAD 301, 302 include a catheter 310 that includes an elongate catheter tube 312 defining one or more lumens 314 extending between a proximal end and a distal end thereof. The proximal end of the catheter tube 312 is operably connected to a hub 316, which in turn is operably connected to one or more extension legs 318. A connector 320, such as a luer connector, is disposed on a proximal end of the extension leg 318, although other connectors, such as spin nuts and the like, are also contemplated.

The hub 316 includes securement features 322, such as, for example, two wings that oppositely extend from the body of the hub 316. It will be appreciated, however, that the size and shape of the securement feature 322 can vary from what is shown and can include various protrusions, abutment surfaces, apertures, or the like and fall within the scope of the present invention. Exemplary securement features and associated securement devices are also shown in U.S. Pat. No. 6,770,055, filed Jun. 29, 2001 and titled “Universal Catheter Anchoring System;” U.S. Pat. No. 9,616,200, filed Dec. 23, 2014 and titled “Intravenous catheter anchoring device;” U.S. Pat. No. 9,694,130, filed Jul. 2, 2012 and titled “Stabilizing device having a snap clamp;” and U.S. Pat. No. 9,480,821 filed Jan. 30, 2012 and titled “Anchoring system for a medical article,” each of which are herein incorporated by reference in their entirety. In an embodiment, each securement wing 322 includes an aperture 324. Note that the hub 316 can also be referred to herein as a “bifurcation hub” depending on the number of fluid passageways included in the embodiment.

In an embodiment, one or more sensors, e.g. sensors 332, 334, are included with the VAD 300 and are collectively referred to herein as a “sensor array” 330. The sensor array 330 can further include a printed circuit board (PCB) 336 that is configured to govern operation of the sensor array 330 and/or store and provide information about the VAD 300 device. The sensor array 330 detects one or more characteristics of the infusion fluid, physiological aspects of the patient, or combinations thereof. In an embodiment the sensor array 330 includes a pressure sensor, ECG sensor, temperature sensor, glucose sensor, oxygen saturation sensor, and the like.

In an embodiment, the PCB 336 includes a microprocessor for governing sensor operation. In an embodiment, the PCB 336 can further include a power source 362 for powering the sensor array 330, though in other embodiments the power source can be remotely disposed from the PCB, and even the VAD 300, as discussed herein. A non-volatile memory storage location, such as flash memory for instance, can also be included on the PCB 336 to enable data to be temporarily or permanently stored thereon. The storage location can be accessible by a user or can be transmitted to a desired location in a manner described herein. In an embodiment, the PCB 336 further includes a communications module, for enabling the PCB 336 to be communicatively coupled with a remote device or location 150. As used herein, “communicatively coupled” includes wired or wireless communication modes. Exemplary remote locations can include the securement device 400, pump 200, handheld devices, smart phones, local area networks (LAN), electronic medical records (EMR) servers, cloud storage facilities, or the like. Exemplary wireless communication modes can include Bluetooth, Wi-Fi, radiofrequency, near-field communication (NFC), or the like.

In an embodiment, the VAD 300 can include a physical, wired connection, e.g. electrical contact 340, to provide power and/or data transmission between the sensor array 330, PCB 336, power source 362 of the VAD 300, securement device 400, pump 200, or combinations thereof. The sensor data can be transmitted from the VAD 300 via a physical connection, such as via a removable physical connection, wires, etc. In an embodiment, sensor data are stored in a memory location included on the PCB 336, or other location on VAD 300. In an embodiment, the PCB 336 includes a clock/timer circuit.

In an embodiment, multiple sensors are included with the VAD 300, though the number, type, size, placement, function, and desired uses of the various sensors can vary from what is shown and described herein. Note that the sensor array 330 can, in one embodiment, include only one sensor. Note also that, where only one of a particular sensor is discussed below, it is appreciated that more than one of a particular type of sensor can be included, in the same or different locations within the VAD 300, or the system 100.

In an embodiment, the sensor array 330 is disposed within the hub 316, which is sized to provide the needed volume for such sensors. Note that the size, shape, and configuration of the hub 316 can vary from what is shown and described in order to house the sensor(s). In other embodiments, the sensor array, or individual sensors of the sensor array 330 can be located in other portions of the VAD 300, including along or at either end of the catheter tube 312, the extension leg(s) 318, or the like. Also note that a variety of sensors for detecting body measurements, physiological aspects of the patient, and/or physical aspects of the VAD 300 can be included with the VAD 300, as discussed herein. Further examples of VAD 300 including sensor arrays 330 are described in U.S. Pat. No. 10,433,790, which is incorporated by reference in its entirety herein.

In an embodiment, as shown in FIGS. 3A-3B, the apertures 324 of the securement wings 322, connectors 320, or combinations thereof include electrical contacts 340 to provide power and/or data communication, to the sensor array 330 and/or PCB 336 of the VAD 300. In an embodiment, an annular electrical contact 340 is included in each aperture 324 of the securement wings 322, with the electrical contacts being operably connected to the PCB 336 and sensor array 330. A securement device, such as the securement device 400 shown in FIGS. 4A-4C, is configured to be placed on the skin of the patient and operably connect with, and secure in place, the VAD 300 once the distal portion of the catheter 312 has been inserted into the patient. To that end, the securement device 400 includes a retainer 454 mounted to an adhesive pad 410, and securement arms 456 that are hinged so as to removably pivot atop the securement wings 322 of the hub 316 (in a snap-fit arrangement) to secure the hub 316 in place.

In an embodiment, the securement device 400 includes additional functionality to provide power and/or data transfer to the sensor array 330 and/or PCB 336. The securement device 400 includes two posts 458, each of which is configured to serve as an electrical contact 460 and each of which is operably connected with a power source 462, for example, a battery. As shown the power source 462 is located on the securement device 400, however in embodiments, the power source can also be located on the VAD 300. The posts 458 are configured to be received within the corresponding apertures 324 of the securement wings 322 such that electrical contact is established with the electrical contacts 340 of the apertures 322. The power source 362 included on the securement device 400 can, in this way, provide electrical power to the sensor array 330 and the PCB 336. Of course, other external power sources can be employed. In an embodiment, the securement device 400 can include a communications module for transmitting sensor data received from the sensor array 330.

FIG. 3B shows an embodiment of a dual-lumen VAD 302. It will be appreciated that additional lumen embodiments, (e.g. triple-quadruple-lumen VAD, etc.) are also contemplated and fall within the scope of the present invention. As with that of FIG. 3A, the VAD 302 shown in FIG. 3B include sensor arrays 330 similar to that shown in FIG. 3A, including corresponding sensors 332, 334, and PCB 336. The electrical contacts 340 for electrical connection with electrical contacts 460 of the securement device 400 (FIGS. 4A-4C) are also shown. Note that each extension leg 318 of the VAD 302 in FIG. 3B includes a corresponding sensor 332 such that data may be sensed in each extension leg. In other embodiments, a sensor and/or PCB is associated with a needle-less injection cap 321 attached to each connector 320, as shown in FIG. 1A. The sensor(s) and PCB(s) in the needle less injection caps can replace or be additional to sensors in other components of the VAD. In other embodiments, more or fewer sensors than what is shown here can be employed for sensing physiological aspects of the patient and/or aspects of the VAD 302 including, for instance, lactic acid sensors, glucose sensors, oxygen sensors, ultrasound componentry, GPS location sensors, temperature sensors, sizing sensors to measure intraluminal diameter, fluid velocity sensors, accelerometers, blood volumetric, cardiac output sensors, etc.

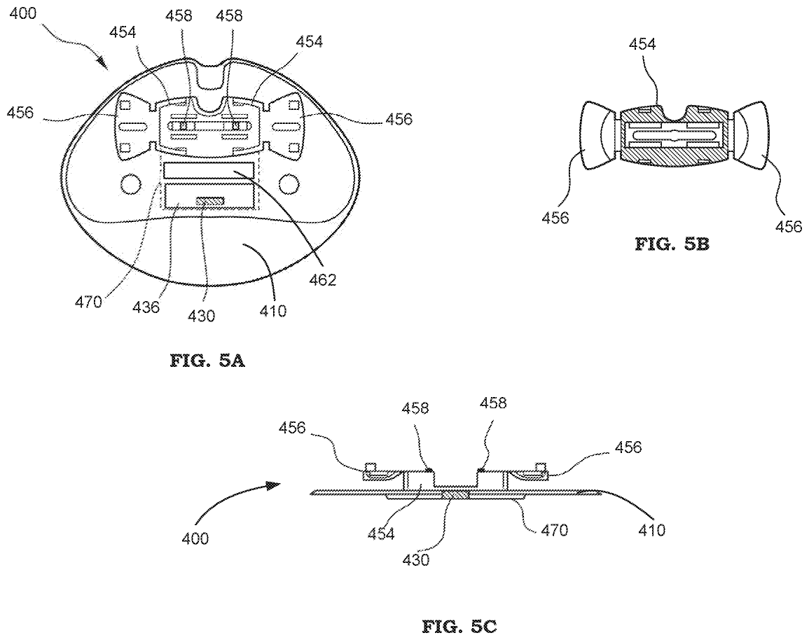

FIGS. 4A-5C depict details of embodiments of a securement device 400. In an embodiment, the securement device includes a pod 470 that includes a sensor array 430, a PCB 436, a power source 462, or combinations thereof. The sensor array 430 of the securement device 400 can include one or more sensors as described herein. The sensor array 430, or portions thereof, can be disposed on a lower surface of the securement device 400. In an embodiment the sensor array 430, or portions thereof, can extend through the retainer 454, anchor pad 410, in order to access a lower surface of the securement device 400. When the securement device 400 is secured to a skin surface of the patient, the sensor array 430 can also contact a skin surface of the patient. The sensor array 430 can detect various physiological characteristics of the patient such as heart rate, body temperature, oxygen saturation, or the like.

One or more of the sensor array 430, PCB 436, power source 462, of the securement device 400 can be coupled with the sensor array 330, PCB 336, or power source 362, of the VAD 300. This can be achieved either wirelessly by way of communication modules located on the respective PCB 336, 436, or by way of physical, wired connections of the apertures 324, posts 458 and associated electrical contacts 340, 460, as described herein. In an embodiment, this eliminates the need for a PCB and/or power source to be disposed on either of the VAD 300 or securement device 400. In an embodiment, one or more of the PCB 336, 436 and sensor array 330, 430 are communicatively coupled with each other and can work in conjunction. In an embodiment, a single power source located on either the VAD 300 or the securement device 400 can power the PCB 336, 436, and sensor array 330, 430 of both the VAD 300 and securement device 400. In an embodiment, a single PCB located on either the VAD 300 or the securement device 400 can govern the sensor array 330, 430 of both the VAD 300 and securement device 400.

In one embodiment, the pod 470 is configured to be removable from the securement device 400, thus enabling it to be reusable with successive securement devices. This may be helpful when the VAD 300 and/or the securement device 400 are changed out. Thus, the pod 470 can be removed from the securement device and placed in another, thus saving resources and cost. Note also that battery and PCB can be disposed in other locations as well. These and other variations are therefore contemplated. Further details regarding a catheter securement device related to those described herein can be found in U.S. Pat. No. 6,770,055, which is incorporated herein by reference in its entirety.

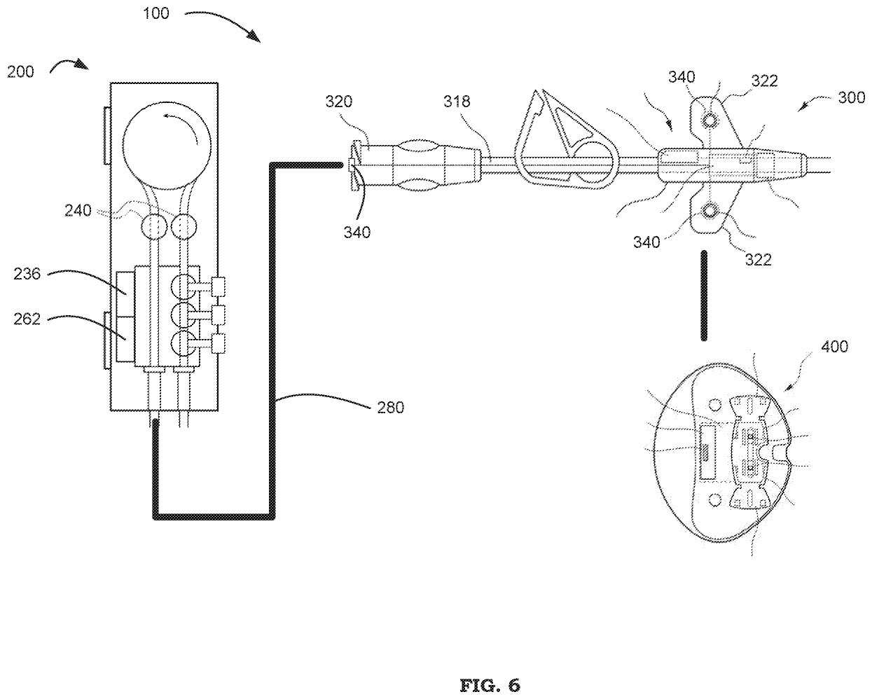

In an embodiment, and as shown in FIG. 6, a PCB 236, power source 262, or combinations thereof can be located on the pump 200 and coupled with the PCB 336, 436 and/or sensor arrays 330, 430 of the VAD 300 and/or securement device 400, or combinations thereof. As described herein, the PCB 236 can include a microprocessor, non-volatile storage, and communications modules to govern any sensor arrays 240, 330, 430, or other PCB 336, 436 coupled thereto, and receive, store and analyze any data received therefrom. In an embodiment, PCB 236 can be communicatively coupled with sensor arrays 240, 330, 430, or other PCB 336, 436 by way of wireless communication, as described herein. In an embodiment, PCB 236 can be communicatively coupled with the sensor array 240, 330, 430, or PCB 336, 436 by way of wired communication. Further, sensor array 330, 430, and PCB 336, 436 can be powered by a power source 262 located on pump 200. As illustrated schematically in FIG. 6, a physical power and/or data connection 280 can extend from the pump 200 to the VAD 300. The connection 280 can either be a separate wire, extending from the pump to the VAD 300, or can be embedded within the wall of the supply lines 122. In the case of the latter, the connection 280 can couple with an electrical contact 340 disposed within the connector 320 and extending through extension leg 318 to couple with electrical contacts 340 of the securement features 322. Accordingly, the PCB 236 can couple with the PCB 436 and sensor array 430 of the securement device by way of apertures 324, posts 458 and associated electrical contacts 340, 460, as described herein.

Advantageously, the reusable components of the system 100, such as the PCB and power source can be located on the pump unit 200 and coupled with the sensor arrays located on the VAD 300 and securement device 400. This reduces the weight of the components attached to the patient, as well as reducing the costs of the disposable VAD/securement components. Similarly, the pump unit 200 is able to accommodate larger power sources, i.e. larger batteries, rechargeable batteries, mains power converters, and the like greatly extending the working life of the system 100. Further, the pump unit 200 is able to accommodate larger PCB components providing greater storage and processing capabilities. Further, in an embodiment where one or more of the power source, PCB, communications module, etc. are located on pump unit 200, expired components, e.g. batteries, can be replaced without disturbing the insertion site.

In an embodiment, the pump unit 200 can modify the output of the pump 220 to accommodate differences if flow mechanics for the system 100. The fluid mechanics, or flow characteristics, of the system 100 can be affected by differences in type of VAD used, type of fluid used, and physiology of the patient. Some of these characteristics can be predetermined while other can vary over time. The pump unit 200 of the system 100 can analyze information from the VAD 300, securement device 400, or sensors 240 within the pump 200 itself to determine the correct output required. Further these flow characteristics can be communicated with a remote locations 150, such as a handheld device, nurses' station, EMR, or the like so that the infusion process can be logged, monitored, and updated. Alerts to a clinician, caregiver, or patent can also be sent according to a desired limit associated with flow characteristics, physiological characteristics, fluid characteristics, VAD characteristics, or a combination of characteristics.

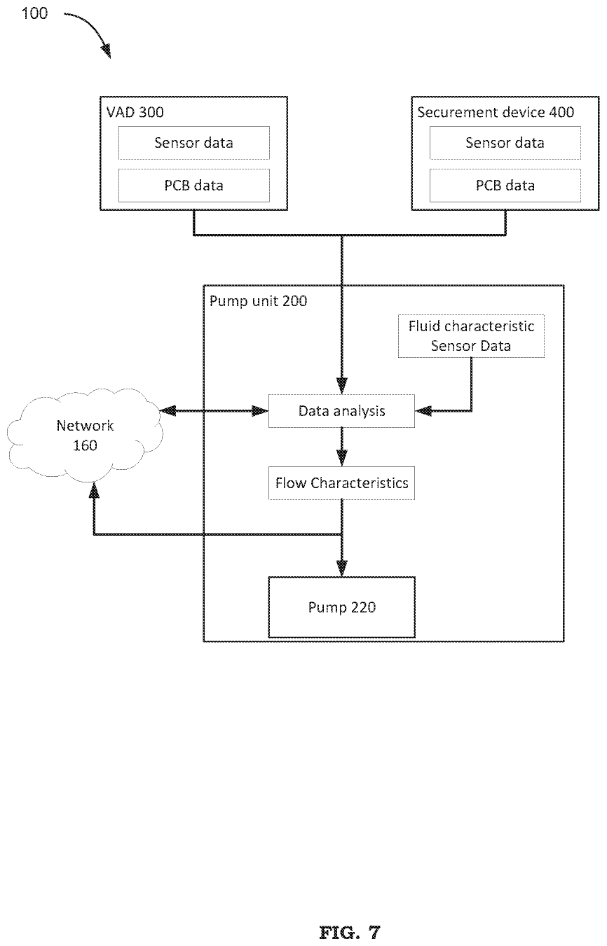

FIG. 7 illustrates a schematic view of the information flow within the system 100, in accordance with an embodiment. Different types of VAD 300, e.g. VAD 301, 302, can be used with the pump unit 200. Each type of VAD defines different flow mechanics depending on the specific characteristics of the VAD. For example, each type of VAD 300 can vary in catheter tube length, number of lumens, luminal cross-sectional area, luminal cross-sectional shape, type of bifurcation hub, catheter tube tip configuration, shape and size of catheter tip openings, etc. Each type of VAD 300 can also vary in the presence or absence of extension legs, number of extension legs, length, cross-sectional area, and cross sectional shape of extension legs, the number and types of connectors, the size, length, and cross-sectional area/shape thereof. Each type of VAD 300 can also vary in the characteristics of the material used, such as flexibility, durometer, elasticity, malleability, etc. Each of the aforementioned characteristics can have an effect on the flow mechanics of a fluid passing through the VAD 300.

As such, each type of VAD 300 can display different flow characteristics. These flow characteristics can be predetermined and stored on the VAD device itself, such as on the non-volatile storage media of the PCB 336. In an embodiment, each type of VAD can be designated a unique identifier, such as an alphanumeric code, icon, or the like. This unique identifier can be stored on the VAD itself, either visibly printed on the outside of the device, stored electronically on the PCB 336, or combinations thereof. This unique identifier can then be retrieved and compared against a database wherein the VAD characteristics can be retrieved.

In an embodiment, the VAD can include interchangeable components. For example, the connectors 320, needle-less injection caps 321, extension legs 318, hub 316, catheter 310, or combinations thereof can be interchangeable. Accordingly, sensors located on the VAD 300 or pump 200, as described herein, can measure key parameters of the different components, or of the fluid flowing through the device. The pump 200 can then analyze the information and derive the flow characteristics of the VAD.

The fluid mechanics of the fluid passing through the system 100 can also vary depending on the type of fluid(s) used. Sensor arrays 240, 330, 430 can measure characteristics of the fluid passing through the system. These fluid characteristics can include the type of fluid(s) used (saline, medications, blood, plasma, etc.), viscosity, concentration, pH, temperature, volume, flow rate, glucose levels, oxygen saturation, electrolyte levels, hematocrit, and the like.

The pump unit 200 can receive additional information from a user regarding the fluid characteristics such as the type of fluid(s) being used, concentrations, volumes, etc. These can be inputted directly to the pump unit 200 or by way of a remote location 150 and network 160. Further, the pump unit 200 can use the fluid characteristics provided to query a database to receive additional fluid characteristics. For example, the user can provide the type of fluid, drug, etc. being administered and the pump unit 200 can query a database, located at a remote location 150 by way of the network 160, to retrieve additional information about the fluid characteristics. In an embodiment the sensor arrays 240, 330, 430 can detect a physiological aspect of the patient. Exemplary physiological aspects include core or peripheral body temperature, oxygen saturation, heart rate, ECG, blood pressure, blood glucose levels, lactate levels, and the like.

Accordingly, the pump unit 200 retrieves and analyzes VAD characteristics, fluid characteristics, physiological characteristics from the sensor arrays 240, 330, 430, or network, and determines the flow characteristics required for the infusion. The pump unit 200 can then modify the pump output accordingly. In an embodiment, the pump unit 200 can determine a schedule to modify the flow characteristics over time during the course of the infusion. For example, different combinations of medications can be applied throughout the course of the infusion and each combination may require different flow rates.

In an embodiment, the pump unit 200 includes a continuous feedback loop to monitor changes in the VAD characteristics, fluid characteristics and physiological characteristics from the sensor arrays 240, 330, 430 and modify the pump output accordingly. For example, the pump unit 200 can detect a change in patient body temperature, heart rate, oxygen saturation of the patient and modify the temperature of the fluid, or the concentration of medications accordingly.

For example, the pump unit 200 can detect a change in fluid pressure of the VAD or time elapsed to determine an occlusion needs clearing or the VAD requires changing. Fluid characteristics, flow characteristics and VAD characteristics can be used to detect an occlusion, a forming occlusion, phlebitis, infiltration, end of therapy, infection, dislodgment of the VAD, leakage, kink, and the like. The pump unit 200 can modify the flow characteristics accordingly, i.e. increase pressure to clear occlusion, or halt fluid flow and provide an alert. The alert can be a visual, auditory, or tactile alert provided by the pump unit 200. In an embodiment, the pump unit 200 can communicate with a remote location, such as a hand held device, smartphone, or nurses station to provide an alert.

In an embodiment, the pump unit 200 can determine that the VAD requires flushing or cleaning. This can be determined by changes in fluid pressure, by time elapsed, or the like. The pump unit can then implement a schedule of pump output changes that clean and flush the VAD. Pump output settings, schedules and the like can be provided to a remote location 150 for further storage, analysis and modification by a user.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. An infusion system, comprising:
a vascular access device (VAD) including one of a first sensor and a first printed circuit board (PCB);
a securement device including one of a second sensor and a second PCB, the securement device configured for securing the VAD to a skin surface of a patient; and
a pump unit in fluid communication with the VAD, wherein the pump unit is configured to:
communicate with at least one of the VAD and the securement device; and
receive information from at least one of the VAD and the securement device in order to modify a flow characteristic of a fluid.

2. The infusion system according to claim 1, wherein the information received from one of the VAD and the securement device includes one of a VAD characteristic, fluid characteristics, and physiological characteristic.

3. The infusion system according to claim 2, wherein the VAD characteristic includes one of sensor data and a unique identifier.

4. The infusion system according to claim 3, wherein the pump unit queries the unique identifier against a database to retrieve additional VAD characteristics.

5. The infusion system according to claim 1, wherein the flow characteristic of the fluid includes one of flow rate, change in flow rate, pressure, and change in pressure.

6. The infusion system according to claim 1, wherein the first and second sensor detects one of body temperature, heart rate, fluid pressure, pH, glucose, and lactate.

7. The infusion system according to claim 1, wherein one of the first and second sensor includes an array of two or more sensors.

8. The infusion system according to claim 1, wherein the pump unit includes a power source operably connected to at least one of the first sensor, the second sensor, the first PCB, and the second PCB.

9. The infusion system according to claim 1, wherein the pump unit is communicatively coupled with one of a remote device and network for sending and receiving information about one of the VAD characteristics, fluid characteristics, physiological characteristics, and flow characteristics.

10. The infusion system according to claim 1, wherein the pump unit is configured to alert one of a clinician, caregiver, or patient according to a desired limit selected from the group consisting of a change in flow characteristics, a change in physiological characteristics, a change in fluid characteristics, a change in VAD characteristics, and combinations thereof.

11. The infusion system according to claim 1, wherein the VAD includes both the first sensor and the first PCB.

12. The infusion system according to claim 1, wherein the securement device includes both the second sensor and the second PCB.

13. The infusion system according to claim 12, wherein the VAD includes both the first sensor and the first PCB.

14. A pump unit for providing a fluid to a vasculature of a patient, the pump unit comprising:
a pump;
a sensor for detecting a fluid characteristic of the fluid;
a printed circuit board (PCB) communicatively coupled with a vascular access device (VAD),
wherein the pump unit is designed to retrieve information from the VAD and the sensor to determine a flow characteristic of the fluid.

15. The pump unit of claim 14, wherein the flow characteristic includes one of flow rate, change in flow rate, pressure, and change in pressure.

16. The pump unit of claim 14, wherein the fluid characteristic includes one of a fluid type, volume, concentration, pH, density, and viscosity.

17. The pump unit of claim 14, further including a securement device for securing an external portion of the VAD to a skin surface of the patient, the securement device communicatively coupled with the VAD and including a sensor for detecting a physiological characteristic of the patient.

18. The pump unit of claim 14, wherein the pump unit provides flow characteristic information to a remote location, the remote location including one of a handheld device, smartphone, laptop computer, server, storage device, patient electronic medical records system, and nurse station.

19. The pump unit of claim 14, wherein the pump unit includes a continuous feedback loop to modify the flow characteristic of the fluid in response to a change in information from one of the VAD and the sensor.

20. The pump unit according to claim 14, wherein the pump unit is further designed to alert one of a clinician, caregiver, or patient according to a desired limit selected from the group consisting of a change in flow characteristics, a change in physiological characteristics, a change in fluid characteristics, a change in VAD characteristics, and combinations thereof.

21. A method of providing dialysis to a patient, comprising:
providing a pump unit, a vascular access device (VAD), and a securement device, one of the VAD and the securement device including a sensor;
accessing a vasculature of a patient using the VAD;
securing an external portion of the VAD to a skin surface of the patient using the securement device;
providing power to the VAD from a power source located on the pump unit;
deriving a VAD characteristic from the VAD;
deriving a fluid characteristic of the fluid from a sensor included in the pump unit;
determining a flow characteristic of the fluid; and
modifying a pump output according to the flow characteristic of the fluid.

22. The method of claim 21, further including a continuous feedback loop wherein in response to a change in one of the VAD characteristic and the fluid characteristic, the pump unit modifies the flow characteristic of the fluid.

23. The method of claim 21, wherein the securement device includes a sensor for detecting a physiological characteristic of the patient, the physiological characteristic including one of heart rate, ECG, oxygen saturation, blood pressure, core body temperature, blood glucose level, and lactate level.

24. The method of claim 23, wherein in response to a change in one of the VAD characteristic, the fluid characteristic, and the physiological characteristic of the patient, the pump unit modifies the flow characteristic of the fluid.

25. The method of claim 21, further comprising alerting one of a clinician, caregiver, or patient according to a desired limit selected from the group consisting of a change in flow characteristics, a change in physiological characteristics, a change in fluid characteristics, a change in VAD characteristics, and combinations thereof.
